(19) United States
(12) Patent Application Publication
(10) Pub. No.: US 2021/0236111 A1
(43) Pub. Date: Aug. 5, 2021

(54) APPLIED TO A SKELETAL MINI ANCHORAGE SITE

(71) Applicant: CAUDURO PARTICIPAÇÕES EIRELI, Araquarl, SC (US)

(72) Inventors: ROBERTO SANTOS CAUDURO, Porto Alegre (BR); Ronaldo Webster, Porto Alegre (BR); Miguel Angelo Ribeiro Scheffer, Porto Alegre (BR)

(21) Appl. No.: 17/064,889

(22) Filed: Oct. 7, 2020

(30) Foreign Application Priority Data
Jan. 31, 2020 (BR) 202020002197-0

(51) Int. Cl. A61B 17/04

(52) U.S. Cl. CPC A61B 17/0401; A61B 2017/0464; A61B 2017/0414

(57) ABSTRACT

AN IMPROVEMENT APPLIED TO A SKELETAL MINI ANCHORAGE SITE in which the use of a modified anchor screwa (1) without a self-perforating tip (2) and without the upper end (5) allows for miniaturized anchorage, eliminating the need to use a prefabricated kit in order to combine quality and cost savings in the anchoring procedure.

### FIELD OF UTILITY MODEL

The present improvement concerns a technical solution presented in the skeletal anchorage system for suture, in particular to a modified screw in order to allow for micro anchorage, resulting in simplification and cost savings for the use of materials required for procedures to reattach tendons, tendon structures and containment of articular discs, but it is not limited to these.

### STATE OF THE ART

The current existing models in the market carry self-perforating anchor screws that are sold in kits comprising the joint sale of prefabricated drivers in which the suture wire and needles are previously incorporated into them, and the whole piece must be disposed of after use. As these screws are self-perforating, they have an end (conical tip) intended for perforation, as well as an upper active tip (an end opposed to the tip) through which the suture wire passes.

In other words, these are single-use, disposable products of a high manufacture value and, consequently, a high sale value, as can be seen in the drawings for PCT US2014062797, titled MODULAR TISSUE REPAIR KIT AND RELATED DEVICES AND METHODS, by SMITH & NEPHEW, INC.

In this type of product, the proposal of anchorage of suture for prosthesis is kept, but the difference lies in the need for acquiring a single piece as a prefabricated kit that is used to secure the anchor to its destination. After use, the orthopedic implant remains attached to the body, and the rest of the kit is disposed of, since it is produced for single use.

In addition, as they have functional ends, with a conical tip and a final end (upper active tip) through which the suture wire is introduced, these products have a greater depth than the one presented in this utility model application. Therefore, the possibilities of applying an anchor are very limited, as this needs a specific bone base for its fixation.

### PRESENTED IMPROVEMENT

In the model presented, there is an improvement that allows for the minimally invasive placement of an implant that serves as a skeletal anchorage site for the suture, allowing a larger number of body parts to receive this type of miniaturized implant.

The main difference lies in the shape of the anchor screw, which in the current disclosure does not have a conical tip and, thus, is not self-perforating; in addition, the upper active tip used to secure the suture wire has also been removed. Accordingly, the size of the screw has been drastically reduced, allowing it to have more possibilities of fixation sites in the body, and only a small amount of bone tissue thickness is required to secure the anchor.

In addition, as the anchor screw is not self-perforating, there is the need for a cavity to be previously created by a rotary perforating instrument, which reduces the chances of rupture of the surrounding bone tissues as it decreases the deployment force and the torsion rate. Also due to the fact that it is not self-perforating, this screw can be placed at the desired depth and even be removed by unscrewing.

In order to further reduce the depth size of the anchor screw, the upper active tip through which the suture wire is typically passed has been removed, and in the model shown, the suture thread passes through an orifice intended for it inside the screw body, which enhances its strength to a great extent. This screw is also manufactured with side channels that allow the wire to pass without being subject to the cutting friction of the pair's threads.

The upper end is developed to allow screwing with a square driver of preferably 1 (one) millimeter, which can be a driver of the most varied shapes, for example, slot, cross, square, hexagon, various polygons, torx, triangle-head, etc.

Therefore, this utility model allows the attachment and function of muscle groups or tendons to be restored, but it is not limited to this, and, consequently, limb movements or muscle functions are restored. The presented screw can have its functions and benefits expanded in terms of shape and size, which is preferably between 3 mm to 20 mm, and areas of applicability, using the same principles, but in other various parts of the body, for example, retention, limitation of range of motion, and positioning of articular disks.

This precision bone anchorage improves the alternatives of tissue fixation for delicate structures in areas under strain. The need for safe bone attachments to soft tissues and tendon skeletal traction always poses specific difficulties.

For example, in the case of hand and foot tendons, the distal bone anchorage structures—the phalanges—are of small size. The same happens to the carpal bones and it's tendon ligaments.

As the suture anchor screw is miniaturized, it can be placed by screwing on delicate bone structures, as in facial surgery, allowing also the anchorage of small articular structures, such as the temporomandibular joint and the soft tissues of the upper, medial and lower third of the face that are asymmetric or drooping.

As it presents multiple usability and is not a prefabricated kit, the surgeon himself will have the option to choose the suture wire he deems the most appropriate for the procedure to be carried out.

As it is a set of reusable instruments, unlike the disposable kits in the current state of the art, the costs of the materials are negligible compared with the benefits and it is easy to use, consequently leading to a lower cost and lower environmental impact.

By avoiding the unnecessary disposal of the materials used for this type of procedure, associated with a high rate of final quality of the mini implant, the present model boils down to an ideal combination of quality and cost savings, without any waste or unnecessary disposal of materials.

Together with the aspects above, a range of suture wires is necessary to connect different types of tissues, and as this is not a prefabricated kit, the presented model provides the surgeon with a free choice and presents this possibility of use, associated with delicacy, strength, inconspicuousness and adjustable osteointegration.

As mentioned above, there is a cost reduction for the entire procedure, which can be ideally intended for the public health system as well as any consumer, for a broader access of the low-income population. These cost savings arise from the effectiveness of the technique, since there is compatibility with materials promptly found in clinics and hospitals, eliminating the need to purchase costly disposable prefabricated kits, enabling a significant cost reduction associated with an increased quality of the final implant. Thus, the inventory needed for the same end activities is drastically reduced.

The present utility model comes down to this set of improvements and benefits.

### BRIEF DESCRIPTION OF FIGURES

The present invention will be fully understood from the detailed description of the present utility model provided below for illustrative but not limitative purposes:

FIG. 1 shows a full side view of the anchor screw (1), also showing the absence of a conical tip (2), the orifice (3) for the suture wire (7) to pass, the side canal (4), and the upper end cavity (5) for fitting a squared shape driver (6).

FIG. 2 shows a perspective view emphasizing the canal (4) for the suture wire (7) to pass and the upper end cavity (5) for fitting the squared shape driver (6).

FIG. 3 shows a side view photo of the anchor screw (1) fitted into the square driver (6) and with the suture wire (7) in an implant prefixation status, passing inside the orifice (3) and adjusted into the side canals (4).

FIG. 4 shows a full view of the non-disposable implant kit (8) with a square driver (6) fitted into the anchor screw (1) and the suture wire (7) already in the implant prefixation position.

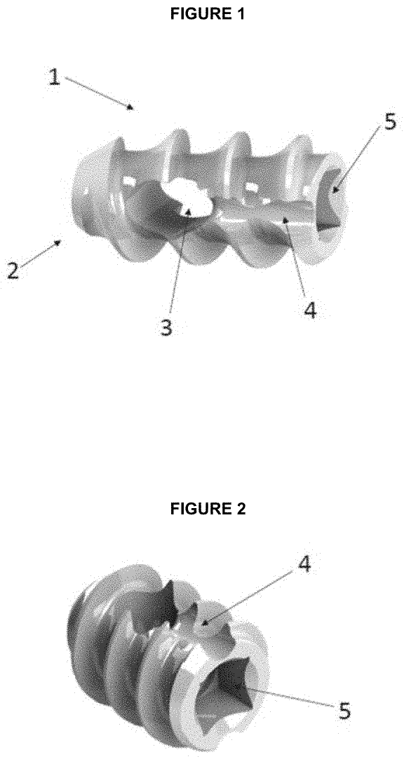

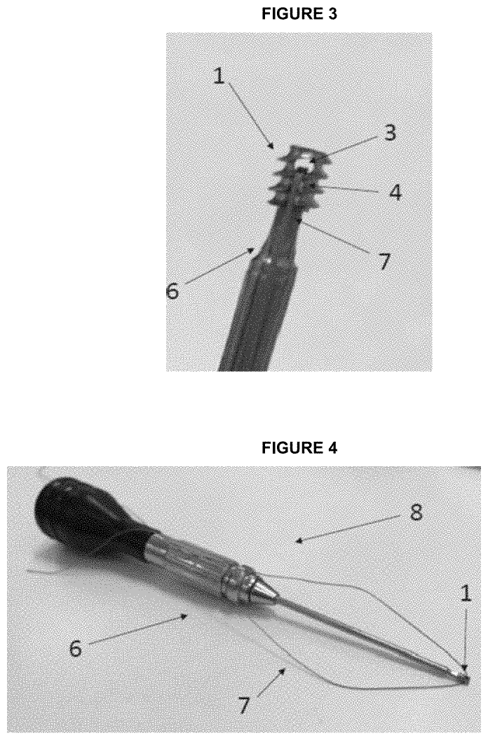

### DETAILED DESCRIPTION OF THE INVENTION

To enable the present anchorage proposal, enough bone tissue thickness must be present for placing the anchor screw (1) into a cavity created by a rotary perforating instrument, for example, a surgical drill. Then the chosen suture wire (7) is passed through the orifice (3) that preferably has a diameter between 0.3 mm and 5 mm, and the suture wire (7) is adjusted into the side canals (4), thus enabling torsion without causing the wire (7) to wrap around the screw. (1).

The anchorage is then screwed using a squared shape driver (6) easily found in the implant market, preferably 1 mm (one millimeter), for the smallest device, this being the critical case, and the driver can be in the most varied shapes, such as, for example, slot, square, hexagon, various polygons, torx, triangle head, etc.

Once the anchor screw (1) is in the desired position, the suture wires (7) are used to retain the desired structures in place, through a new insertion, and the passage is allowed by side canals (4) of preferably 0.3 mm to 3 mm.

Thus, the use of the anchor screw (1) without a self-perforating tip (2) and without the upper end (5) allows for the miniaturized anchorage, eliminating the need to use a prefabricated kit, and the cavity must be created by a piercing instrument.

In short, the improvements presented to the miniaturized anchor screw (1) represent greater safety and customization for interventions with fewer scars and for general orthopedic surgeries of small fragments and muscle-tendon reattachments.

Thus, when a professional uses the present miniaturized anchor screw (1) to anchor the desired tissue, it has improvements in the anchorage assembly that go beyond the convenience of handling and result in work completed with greater efficiency, durability and security, associated with a significant reduction in current market costs.

1. Improvement applied to a skeletal mini anchorage site comprising a modified miniaturized anchor screw (1) characterized in that it is manufactured without a self-perforating conical tip (2) and does not have an upper end (5) for passing the suture wire (7), which is replaced with the fitting portion of a squared shape driver (6) and presents an orifice (3) at the internal end and side canals (4) for the suture wire (7) to pass.
